(19) United States
(12) Patent Application Publication
(10) Pub. No.: US 2019/0062791 A1
(43) Pub. Date: Feb. 28, 2019

(54) PRODUCTION OF BIOFUELS, BIOCHEMICALS, AND MICROBIAL BIOMASS FROM CELLULOSIC PULP

(71) Applicant: DOMTAR PAPER COMPANY, LLC, Fort Mill, SC (US)

(72) Inventor: Neelakantam V. Narendranath, Fort Mill, SC (US)

(21) Appl. No.: 16/115,163

(22) Filed: Aug. 28, 2018

Related U.S. Application Data
(60) Provisional application No. 62/551,458, filed on Aug. 29, 2017.

(51) Int. Cl. C12P 7/10; C12P 7/54; C12P 7/48

(52) U.S. Cl. CPC C12P 7/10; C12P 7/54; C12P 2203/00; C12Y 302/01021; C12P 7/48

(57) ABSTRACT

Methods for production of alcohol, biomolecules, and/or microbial biomass using cellulosic pulp as a feedstock are provided. Cellulosic pulp is contacted with an enzyme composition to produce fermentable sugars, which are used as a carbon source for a microorganism to produce alcohols or biochemicals by fermentation and/or to produce only microbial biomass.

### CROSS REFERENCE TO RELATED APPLICATIONS

This application claims the benefit of U.S. Provisional Patent Application No. 62/551,458, filed Aug. 29, 2017, the contents of which application are hereby incorporated by reference.

### FIELD OF INVENTION

The present invention relates generally to systems and methods for producing ethanol, biochemicals, and/or biomass using cellulosic pulp as a carbon source for propagation of microorganisms. The cellulosic pulp used may be derived from pulping processes, such as kraft or sulfite pulping, that are commonly used in making paper. The cellulosic pulp is hydrolyzed by enzymes to produce fermentable sugars, which are used to feed microorganisms that produce biomass, ethanol, and/or other biochemicals using the fermentable sugars as a carbon source.

### DESCRIPTION OF RELATED ART

The pulp and paper industry has always operated as a biorefinery, producing fibers, chemicals such as turpentine and tall oil, the energy needed for the process, and even ethanol (such as from spent sulfite liquor) from lignocellulosic biomass. Processes to separate the C5 sugars (e.g., xylose) from spent sulfite liquor and make xylitol have also been employed. However, in the modern kraft mills, not much attention has been given to the potential fermentation of any of the intermediate pulp streams to either ethanol or other biochemicals. As long as paper was profitable there was not much of a need to develop alternative uses for the intermediate products of kraft pulping. However, due to emergence of electronic documentation, the demand for fine commercial paper has been decreasing. This has led to a need to develop alternative markets for pulp made from both hardwood and softwood.

Production of ethanol and other biochemicals is a significant potential use for intermediate pulp products from paper mills. To date, the lignocellulosic ethanol industry has concentrated predominantly on feedstocks such as corn stover, wheat straw, agricultural residue, switchgrass, and various other grasses. The major obstacles for commercial ethanol production from these feedstocks include insufficient separation of cellulose and lignin, formation of byproducts that inhibit downstream fermentation, high use of chemicals and/or energy, and high capital costs for pretreatment facilities. Therefore, alternative processes for production of ethanol and other end products from lignocellulosic biomass are needed.

### SUMMARY

This disclosure includes processes that meet the needs identified above. In particular, processes are disclosed that use intermediate pulp products from processes commonly used in the paper industry as a feedstock for production of ethanol and other end products such as biochemicals and microbial biomass. The disclosed processes can thus provide an alternative use for intermediate pulp products produced in the paper industry. In addition, the disclosed processes address many of the problems associated with production of end products from lignocellulosic biomass commonly used in the bioethanol industry. Pulp from kraft, sulfite, and soda mills are essentially pretreated lignocellulosic biomass and already have a substantial portion of the lignin removed. Therefore, the cellulose in the pulp is readily accessible to enzymes and can be efficiently hydrolyzed to produce fermentable sugars. In addition, excess quantities of such pulp can readily be produced in paper making processes, making its use economically efficient. Having this alternative source of feedstock for ethanol production avoids the necessity of making the expensive capital investments required for new pretreatment facilities to process grasses and other lignocellulosic feedstocks. The processes disclosed herein thus provide an economical way to produce ethanol and other end products from lignocellulosic biomass.

In one embodiment, a method of producing an end product from pulp is disclosed. The method comprises (a) obtaining an aqueous slurry comprising 8 to 12% by weight of cellulosic pulp; (b) contacting the cellulosic pulp with an enzyme composition to produce fermentable sugars, wherein the aqueous slurry is at a temperature between 45 and 60° C. and a pH between 4.5 and 6; and (c) using the fermentable sugars as a carbon source for a microorganism to produce an end product. As used herein, the percentage by weight of cellulosic pulp in an aqueous slurry is the dry weight of the solids in the cellulosic pulp divided by the total weight of the aqueous slurry. In some embodiments, the cellulosic pulp in the aqueous slurry is brownstock pulp, oxygen delignified pulp, or bleached pulp from a kraft pulping process, a sulfite pulping process, or a soda pulping process. In some embodiments, the cellulosic pulp is an intermediate product in a paper making process. In some embodiments, the cellulosic pulp is washed before it is added to the aqueous slurry. In some embodiments, the cellulosic pulp is not washed before it is added to the aqueous slurry. In some embodiments, the method further comprises adjusting the temperature of the aqueous slurry to between 25 and 38° C. before or during step (c).

In some embodiments, the end product of the process is ethanol. In some embodiments, step (c) further comprises fermentation of the fermentable sugars by the microorganism to produce ethanol. In some embodiments, such fermentation produces ethanol to a concentration of at least 3% w/v or between 3 and 6% w/v.

In some embodiments, the end product of the process is a biochemical. In some embodiments, the biochemical is acetic acid, lactic acid, succinic acid, citric acid, gluconic acid, L-ascorbic acid, adipic acid, or itaconic acid. In some embodiments, step (c) further comprises fermentation of the fermentable sugars by the microorganism to produce the biochemical. In some embodiments, such fermentation produces the biochemical to a concentration of at least 3% w/v or between 3 and 6% w/v. In some embodiments, the fermentation takes place under substantially anaerobic conditions.

In some embodiments, the end product is microbial biomass comprising the microorganism. In some embodiments, the microorganism is yeast, algae, or bacteria. In some embodiments, the microbial biomass is produced by aerobic propagation. In some embodiments, the microorganisms are propagated without fermentation. In some embodiments, substantially no ethanol is produced by the microorganism during step (c), wherein the microorganism is yeast, including Saccharomyces cerevisiae. In some embodiments, no more than 0.1 g/L of ethanol is produced by the microorganism during step (c). In some embodiments, step (c) results in an increase of the biomass of the microorganism by at least 10, 25, or 50 fold or by between 25 and 500 fold.

In some embodiments, the enzymatic hydrolysis of the cellulosic pulp continues during at least a portion of step (c). In such embodiments, growth of the microorganism and production of the end product, which may be accomplished by fermentation of the fermentable sugars, can happen simultaneously with enzymatic hydrolysis of the cellulosic pulp in the aqueous slurry.

In some embodiments, the method further comprises separating the fermentable sugars from solids in the aqueous slurry before step (c). In some embodiments, this is accomplished by filtration. The separated fermentable sugars can then be used as a carbon source for propagation of the microorganism in step (c). In some embodiments, the separated fermentable sugars are added to an aqueous propagation medium comprising the microorganism. In some embodiments, the microorganism is added directly to the solution of separated fermentable sugars.

In some embodiments, step (c) further comprises providing a nitrogen source to the microorganism. In some embodiments, the nitrogen source is urea. In some embodiments, the urea is added to a concentration of between 0.3 and 1 g/L before or during step (c).

In some embodiments, the pH of the aqueous slurry is monitored and adjusted to a pH between 5 and 6 or to a pH of about 5.5 during or before step (b) and/or step (c).

In some embodiments, the fermentable sugars comprise glucose and/or xylose. In some embodiments, contacting the cellulosic pulp with the enzyme composition results in the production of glucose to a concentration of at least 5% w/v or between 5 and 11% w/v in the aqueous slurry. In some embodiments, the microorganism propagated in step (c) is capable of metabolizing xylose and/or other five-carbon sugars.

In some embodiments, the total weight of the enzyme composition of step (b) is between 3 and 9% of the dry weight of the cellulosic pulp in the aqueous slurry. In some embodiments, the enzyme composition comprises one or more enzymes selected from the group of enzymes consisting of: an endoglucanase, a cellobiohydrolase, a xylanase, and a beta-glucosidase. In some embodiments, the enzymes are added as part of a cocktail. In some embodiments, the one or more enzymes are present in the propagation medium at a total enzyme concentration of about 0.03 to 0.09 grams of enzyme per gram of cellulosic pulp on a dry weight basis. In some embodiments, the enzyme composition is CTec3 enzyme cocktail from Novozymes. The concentrations of individual enzymes can be adjusted and optimized to provide for production of desired fermentable sugars at desired rates. The temperature and pH of the propagation medium may also affect the rate of hydrolysis and can be adjusted to provide for production of fermentable sugars at a desired rate.

In some embodiments, the method further comprises producing the cellulosic pulp to be included in the aqueous slurry of step (a). In some embodiments, the pulp is produced from hard wood, soft wood, switchgrass, elephant grass, corn stover, wheat straw, or bagasse. In some embodiments, the pulp is produced by kraft pulping, sulfite pulping, or soda pulping. In some embodiments, the kappa number of the pulp is between about 15 and 100 or between 35 and 75.

The terms “a” and “an” are defined as one or more unless this disclosure explicitly requires otherwise.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “substantially,” “about,” and “approximately” are defined as largely but not necessarily wholly what is specified—and includes what is specified; e.g., substantially 90 degrees includes 90 degrees and substantially parallel includes parallel—as understood by a person of ordinary skill in the art. In any disclosed embodiment, the terms “about” and “approximately” may be substituted with “within [a percentage] of” what is specified, where the percentage includes 0.1, 1, 5, and 10 percent.

Any embodiment of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. For example, the methods of introducing substances into cells disclosed herein can “comprise,” “consist essentially of,” or “consist of” particular components, compositions, ingredients, etc. disclosed throughout the specification.

Other objects, features and advantages of the present invention will become apparent from the following figures and detailed description. It should be understood, however, that the figures and detailed description, while indicating specific embodiments of the invention, are given by way of illustration only and are not meant to be limiting. Additionally, it is contemplated that changes and modifications within the spirit and scope of the invention will become apparent to those skilled in the art from this detailed description.

The feature or features of one embodiment may be applied to other embodiments, even though not described or illustrated, unless expressly prohibited by this disclosure or the nature of the embodiments. Some details associated with the embodiments described above and others are described below.

### BRIEF DESCRIPTION OF THE DRAWINGS

FIG. 1 is a block flow diagram of a process showing use of pulp from various stages of the pulping process to produce sugars by enzymatic hydrolysis and subsequently ethanol by fermentation.

FIG. 2 is block flow diagram of a process showing use of pulp from various stages of the pulping process to produce sugars by enzymatic hydrolysis and subsequently ethanol by fermentation. A liquefaction step is included prior to enzymatic hydrolysis.

FIG. 3 is a block flow diagram of a process showing use of pulp from various stages of the pulping process to produce sugars by enzymatic hydrolysis and subsequently microbial biomass via aerobic fermentation. A liquefaction step (optional in this case) is included prior to enzymatic hydrolysis.

FIGS. 4A-B show results from saccharification of brownstock pulp (FIG. 4A) and bleached pulp (FIG. 4B). Concentrations of glucose produced are shown for the indicated enzyme concentration levels.

FIG. 5 shows the concentrations of ethanol produced from fermentation of brownstock and bleached pulp at the indicated levels of dry solids (% DS).

FIGS. 6A-B show concentrations of glucose and ethanol produced from a process employing simultaneous saccharification and fermentation from brownstock pulp (FIG. 6A) and from bleached pulp (FIG. 6B) at the indicated enzyme concentrations. Glucose concentrations are represented by the dotted lines, and ethanol concentrations are represented by the solid lines.

### DETAILED DESCRIPTION OF ILLUSTRATIVE EMBODIMENTS

The methods disclosed herein efficiently produce an end product using cellulosic pulp as a carbon source for microbial growth and metabolism. The end product may be alcohol, a biochemical, or microbial biomass. The end product is produced by a microorganism using fermentable sugars derived from enzymatic hydrolysis of the cellulosic pulp as a carbon source. The conditions in which the fermentable sugars and end products are produced affect the efficiency of the process. These and other aspects of the disclosed method will be described in greater detail below.

### A. Cellulosic Pulp

In certain embodiments, methods disclosed herein use cellulosic pulp as a feedstock for the production of end products by a microorganism. The cellulosic pulp can come from a variety of sources. In some embodiments, the cellulosic pulp is an intermediate product in a paper-making process. For example, the cellulosic pulp may be pulp made in a kraft, sulfite, or soda pulping process. Pulp from various stages of these processes can be used.

Pulping processes aim to break down the bulk structure of lignocellulosic biomass to separate cellulose fibers from lignin and hemicelluloses in the lignocellulosic biomass. The lignocellulosic feedstock can be a variety of plant materials, including stems, leaves, hulls, husks, cobs, branches, and wood of plants. The lignocellulosic feedstock can be derived from agricultural residue, herbaceous material (including energy crops), municipal solid waste, pulp and paper mill residue, waste paper, and wood (including forestry residue). The lignocellulosic feedstock may include without limitation any of the following: arundo, bagasse, bamboo, corn cob, corn fiber, corn stover, miscanthus, orange peel, rice straw, switchgrass, wheat straw, aspen, eucalyptus, fir, pine, poplar, spruce, or willow.

A typical pulping process in the paper industry is as follows: The lignocellulosic feedstock is first mechanically crushed or cut into smaller pieces to allow pulping liquor to penetrate the pieces completely. The pieces are then mixed with an aqueous solution of pulping chemicals (liquor) and heated under high pressure for several hours. The resulting pulp is referred to as brownstock pulp. The brownstock pulp is then washed, such as by countercurrent flow, which removes the spent liquor, leaving washed brownstock pulp, which typically comprises between about 70 and 80% cellulose fiber on a dry weight basis and about 4-5% lignin on a dry weight basis. The washed brownstock is then treated by oxygen delignification to remove more lignin. This process involves treatment of the pulp with oxygen in a pressurized vessel at elevated temperatures in an alkaline environment. The lignin content of the pulp can be reduced by 40 to 70% during this process. The oxygen delignified pulp is then bleached using chemicals such as chlorine, chlorine dioxide, sodium hypochlorite, ozone, and/or hydrogen peroxide to further reduce the lignin content of the bleached pulp to about 0.1% or less on a dry weight basis.

The cellulosic pulp at any stage of the above-described pulping process can be used in the methods disclosed herein as a source of fermentable sugars for the production of end products such as microbial biomass or alcohol and other biochemicals. The methods disclosed herein can use, for example, brownstock pulp, washed brownstock pulp, oxygen delignified pulp, bleached pulp, or a combination thereof. The pulp can be from a kraft, sulfite, or soda pulping process.

In some embodiments, the cellulosic pulp comprises at least about, at most about, or about 50, 55, 60, 65, 70, 75, 80, 85, or 90% cellulose on a dry weight basis, or any range derivable therein. In some embodiments, the cellulosic pulp comprises at least about, at most about, or about 0.1, 0.5, 1, 2, 3, 4, 5, 6, 7, 8, 9, or 10% lignin on a dry weight basis, or any range derivable therein.

In some embodiments of the methods disclosed herein, the cellulosic pulp is comprised in an aqueous slurry in which the hydrolysis of the cellulose is carried out. The proportion of cellulosic pulp solids in the slurry can be adjusted to provide for optimal hydrolysis efficiency. In some embodiments, the aqueous slurry comprises at least about, at most about, or about 1, 2, 3, 4, 5, 6, 7, 8, 9, 10, 11, 12, 13, 15, 16, 17, 18, 19, 20, 21, 22, 23, 24, or 25% by weight of cellulosic pulp solids, or any range derivable therein.

### B. Enzymatic Hydrolysis

Methods described herein employ enzymes to convert polysaccharides and oligosaccharides in cellulosic pulp to monosaccharides (e.g., glucose), also referred to herein as “fermentable sugars,” that can be taken up and metabolized by a microorganism. The enzymes may be cellulolytic and/or hemicellulolytic enzymes (i.e., cellulases and/or hemicellulases) and may include, for example, one or more of the following types of enzymes, alone or in combination: endoglucanases, cellobiohydrolases, beta-glucosidases, glucoside hydrolases, acetylmannan esterases, acetylxylan esterases, arabinases, arabinofuranosidases, coumaric acid esterases, feruloyl esterases, galactosidases, glucuronidases, glucuronyl esterases, mannases, mannosidases, oxidoreductases, xylanases, xylosidases, and beta-xylosidases. The enzymes may comprise one or more of cellobiohydrolase I, cellobiohydrolase II, endo-1,4-β-glucanase, exo-1,4-β-glucanase, and 1,4-β-glucosidase.

In one aspect, the enzymes comprise a commercial enzyme preparation or enzyme cocktail. Examples of commercial enzyme preparations suitable for use in the disclosed methods include, for example, CELLIC® CTec (Novozymes A/S), CELLIC® CTec2 (Novozymes A/S), CELLIC® CTec3 (Novozymes A/S), CELLUCLAST® (Novozymes A/S), NOVOZYM™ 188 (Novozymes A/S), SPEZYMETM CP (Genencor Int.), ACCELLERASE™ TRIO (DuPont), FILTRASE® NL (DSM); METHAPLUS® S/L 100 (DSM), ROHAMENT™ 7069 W (Rohm GmbH), or ALTERNAFUEL® CMAX3™ (Dyadic International, Inc.).

In some embodiments, the cellulolytic enzymes are present in the aqueous slurry at a total enzyme concentration of about 0.05 to 0.7 grams of enzyme per gram of cellulosic pulp solids in the propagation medium. In some embodiments, the total enzyme concentration is about 0.03 to 0.09 grams of enzyme per gram of cellulosic pulp in the propagation medium. In some embodiments, the total enzyme concentration is at least about, at most about, or is about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.90 grams of enzyme per gram of cellulosic pulp in the aqueous slurry, or is between any two of these values. In some embodiments, 87.5 to 1225 biomass hydrolysis units (“BHU(2)”) of enzymes are present in the aqueous slurry. In some embodiments, 430 to 615 BHU(2) of enzymes are present in the aqueous slurry. In some embodiments, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, or 1250 BHU(2) of enzymes are present in the aqueous slurry, or between any two of these values.

In some embodiments, the concentration of an individual enzyme is at least about, at most about, or is about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.90 grams of enzyme per gram of dry cellulosic pulp in the aqueous slurry, or is between any two of these values. These concentration values can be the concentration of any one of an endoglucanase, cellobiohydrolase, beta-glucosidases, xylanase, and/or beta-xylosidase in the aqueous slurry. These concentration values can be the concentration of cellobiohydrolase I, cellobiohydrolase II, endo-1,4-β-glucanase, exo-1,4-β-glucanase, and/or 1,4-β-glucosidase, or other enzymes disclosed herein in the aqueous slurry. Each of these enzymes may be present at one of the above concentration values in any combination with one or more of any other cellulolytic enzyme.

The temperature of the aqueous slurry during enzymatic hydrolysis may be chosen to maximize the rate of hydrolysis and production of the fermentable sugars. In some embodiments, the temperature of the aqueous slurry is maintained at 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65° C., or any range derivable therein, for at least a portion of the time in which the enzymatic hydrolysis is taking place, or for the entirety of such time.

In some embodiments, enzymatic hydrolysis is performed separately from microbial fermentation and production of end products. This can be done, for example, by allowing the enzymatic hydrolysis to proceed to completion before any microbes are added to the slurry. For example, the hydrolysis may continue for at least 12, 24, 36, 48, 60, 72, 84, 96, 108, or 120 hours until the enzymes stop producing additional fermentable sugars from the cellulosic pulp. This can be monitored by periodically taking samples and determining concentrations of fermentable sugars like glucose. Additionally or alternatively, the fermentable sugars produced during enzymatic hydrolysis can be physically separated from solids in the slurry after hydrolysis has been partially or fully completed. The fermentable sugars can then be fed to microorganisms for production of the desired end product.

Hydrolysis may also continue simultaneously with microbial fermentation and production of the desired end product. One way this can be done is for the microorganism to be added to the slurry before the enzymes have completed hydrolysis of the cellulosic pulp. For example, microorganisms can be added to the aqueous slurry at the same time as the enzymes. Alternatively, the microorganisms can be added to the aqueous slurry some time after the enzymes are added, giving the enzymes time to produce sufficient fermentable sugars to support microbial growth and fermentation. In such embodiments, the enzymatic hydrolysis may be allowed to continue for at least about, at most about, or about 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, or 72 hours, or any range derivable therein, before the microorganisms are added to the slurry. The temperature of the slurry during the enzymatic hydrolysis that takes place before addition of microorganisms may be different than the temperature after the microorganisms are added. This may be done, for example, when the optimal temperature for enzymatic hydrolysis is different from the optimal temperature for the microorganism. In some embodiments, the temperature after addition of enzymes but before addition of the microorganism is at least about, at most about, or about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65° C., or any range derivable therein. After addition of the microorganism, the temperature may be maintained at the same temperature or may be increased or decreased to be at least about, at most about, or about 15, 16, 17, 18, 19, 20, 21, 22, 23, 24, 25, 26, 27, 28, 29, 30, 31, 32, 33, 34, 35, 36, 37, 38, 39, 40, 40, 41, 42, 43, 44, 45, 46, 47, 48, 49, 50, 51, 52, 53, 54, 55, 56, 57, 58, 59, 60, 61, 62, 63, 64, or 65° C., or any range derivable therein. Enzymatic hydrolysis of the cellulosic pulp may continue after addition of the microorganism and change of temperature, if any, but may in some instances happen at a lower rate than at the higher temperature.

Enzymatic hydrolysis of cellulosic pulp and production of end products by microorganisms can both be influenced by the pH of the medium. In some embodiments, the pH of the aqueous slurry during enzymatic hydrolysis is in a range from about 4.0 to 7.0, from about 5.0 to about 6.0, or from about 5.3 to about 5.7. In some embodiments, the pH during enzymatic hydrolysis is at least about, at most about, or about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0, or is between any two of these values. In some embodiments, the pH during production of end product by the microorganisim, whether it be performed at the same time as or separately from enzymatic hydrolysis, is in a range from about 4.0 to 7.0, from about 5.0 to about 6.0, or from about 5.3 to about 5.7. In some embodiments, the pH during production of end product by the microorganism is at least about, at most about, or about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0, or is between any two of these values.

In some embodiments, enzymatic hydrolysis of the cellulosic pulp results in the production of glucose to a concentration of at least about 1, 2, 3, 4, 5, 6, 7, 8, 9, 10, 11, 12, 13, 14 or 15% w/v in the aqueous slurry, or between any two of those values.

### C. Production of End Products

The methods disclosed herein can be used to make a variety of end products via microbial propagation or fermentation. Such microbial processes may include both anaerobic (fermentative) and aerobic growth (propagation) of the microorganism using sugars produced by enzymatic hydrolysis of the cellulosic pulp, along with sugars that may be present in the cellulosic pulp before enzymatic hydrolysis, as a carbon source. The particular end product produced depends on a variety of factors, including growth conditions and the type of microorganism used. In some embodiments, the end product is ethanol, a biochemical such as an organic acid, or microbial biomass.

### 1. Ethanol

Production of ethanol can be accomplished by several organisms by methods known in the art. Ethanol-producing microorganisms include Saccharomyces cerevisiae, Saccharomyces uvarum, Saccharomyces carlsbergensis, Saccharomyces chevalieri, Candida krusei, Candida guilliermondii Candida tropicalis, Candida diddensiae, Candida fabianii, Candida intermedia, Candida maltosa, Candida santamariae, Candida colliculosa, Pichia membranaefaciens, Cryptococcus kuetzingii, Hansenula polymorpha, Kloeckera corticis, Rhodotorula pallida, Rhodotorula rubra, Rhodotorula minuta, Torulopsis norvegica, and Trichosporon cutaneum. In some embodiments, the ethanol-producing microorganisms are genetically modified to broaden the range of sugars that they are able to metabolize and use for the production of ethanol. For example, in some embodiments, the microorganism used to produce ethanol is a Saccharomyces cerevisiae strain genetically modified to be able to metabolize five-carbon sugars such as xylose.

In some embodiments, the microbial fermentation of sugars derived from the cellulosic pulp results in the production of ethanol to a concentration of at least about or about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10% w/v, or between any two of these values.

### 2. Organic Acids

Many different organic acids can be made by microorganisms using sugars derived from cellulosic pulp according to methods known in the art. As non-limiting examples, such organic acids may include acetic acid, lactic acid, succinic acid, citric acid, gluconic acid, L-ascorbic acid, or itaconic acid. Methods of producing these organic acids by microorganisms are known. For example, acetic acid can be produced by several Gluconobacter and Acetobacter species. Lactic acid can be produced by several Lactobacillus species. Gluconic acid can be produced by several bacteria and fungi, including bacterial species of the genera Gluconobacter, Acetobacter, Pseudomonas, and Vibrio and fungal species of the genera Aspergillus, Penicillium, and Gliocladium. Citric acid can be produced by Candida catenula, Candida guilliermondii, and Corynebacterium species, as well as mold fungi from the genus Aspergillus.

In some embodiments, the fermentation of sugars derived from the cellulosic pulp results in the production of organic acids to a concentration of at least about or about 0.5, 1.0, 1.5, 2.0, 2.5, 3.0, 3.5, 4.0, 4.5, 5.0, 5.5, 6.0, 6.5, or 7.0% w/v, or between any two of these values.

### 3. Microbial Biomass

The disclosed methods can be used for production of a variety of useful microorganisms. For example, the methods can be used to produce biomass of any type of yeast used for ethanol production, baking, wine making, distilling, or animal feed. The yeast produced by the methods disclosed herein may be, for example, strains of Saccharomyces, Candida, Torula, and Kluyveromyces genera. In some embodiments, the yeast comprise Saccharomyces cerevisiae, Candida sonorensis, Candida utilis, or Kluyveromyces marxianus. The yeast may be wild-type or may have genetic modifications. Examples of genetic modifications include, for example, causing yeast to express enzymes that enable them to metabolize five-carbon sugars such as xylose. Yeast may also be modified to express cellulolytic enzymes.

The microbial biomass can be used as single-cell proteins, such as those that are commonly used to supplement animal feed. Microbial species useful as single-cell protein include, for example, Saccharomyces cerevisiae, Pichia pastoris, Candida utilis, Geotrichum candidum, Aspergillus oryzae, Fusarium venanatum, Sclerotium rolfsii, Polyporus sp., Trichoderma sp., Scytalidium acidophilum, and Rhodobacter capsulatus.

The microbial biomass produced according to the methods disclosed herein may also be heterotrophic algae, such as those used in aquaculture feed. These algae may include, for example, algae of the Tetraselmis, Schizochytrium, and Chlorella genera.

In some embodiments, propagating the microorganism results in an increase in biomass of the microorganisim of at least at least about 10, 15, 20, 25, 30, 35, 40, 45, 50, 55, 60, 65, 70, 75, 80, 85, 90, 95, 100, 105, 110, 115, 120, 125, 130, 135, 140, 145, 150, 200, 250, 300, 350, 400, 450, or 500-fold, or between any two of those values. In some embodiments, when propagating a microorganism that has the ability to produce ethanol, such as Saccharomyces cerevisiae, such propagation is carried out under conditions that lead to minimal or no production of ethanol, or production of ethanol to a concentration no greater than 0.01, 0.1, 0.2, 0.3, 0.4, or 0.5 g/L.

### D. Illustrative Embodiments

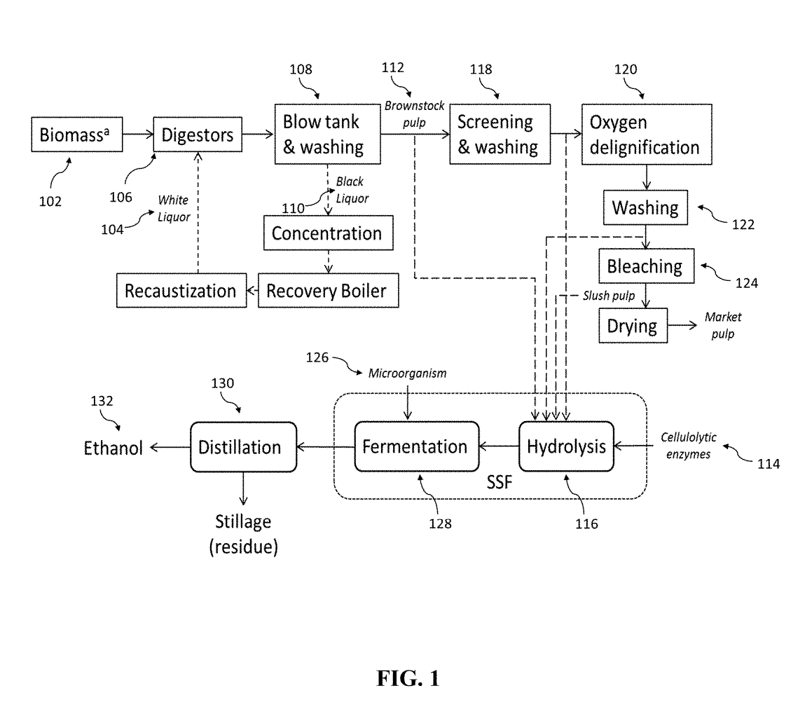

FIG. 1 shows a block flow diagram of a non-limiting embodiment of a method of producing ethanol from cellulosic pulp. This embodiment shows production of cellulosic pulp by kraft pulping, but similar ethanol production processes can be performed using cellulosic pulp from other pulping processes such as sulfite pulping and soda pulping. The process starts with combining lignocellulosic biomass 102 with white liquor 104 in digestors 106. In the illustrated embodiment, the source of the lignocellulosic biomass 102 can be hardwood, softwood, wood residue, or tree thinnings/trimmings. In the digestors 106, the mixture of the lignocellulosic biomass 102 and white liquor 104 is heated under high pressure for sufficient time to break down the structure of the lignocellulosic biomass and release cellulose fiber. The digested biomass 102 is then transferred to a blow tank 108, where it is washed and separated from the black liquor 110. The remaining solids are washed brownstock pulp 112. The washed brownstock pulp 112 is then either mixed with cellulolytic enzymes 114 for hydrolysis 116 of the cellulose fibers in the washed brownstock pulp 112 or processed further before hydrolysis 116 is performed. If the washed brownstock pulp 112 is to be processed further, it is subjected to screening and washing 118, oxygen delignification 120, further washing 122, and bleaching 124. After each of these steps, the cellulosic pulp can be mixed with cellulolytic enzymes 114 for hydrolysis 116, as shown by the dashed lines leading to hydrolysis 116. In the illustrated embodiment, hydrolysis 116 is carried out between 50 and 65° C. at a pH of 4 to 5.5. After a period of hydrolysis 116, but before the cellulosic pulp is completely hydrolyzed, an ethanol-producing microorganism 126 is added, and fermentation 128 is carried out to produce ethanol. In the illustrated embodiment, the fermentation 128 is carried out at 30 to 35° C. at a pH from 4 to 6. At the same time, hydrolysis 116 continues, producing more fermentable sugars from the cellulosic pulp to feed the microorganism 126. After distillation 130, the ethanol end product 132 is collected.

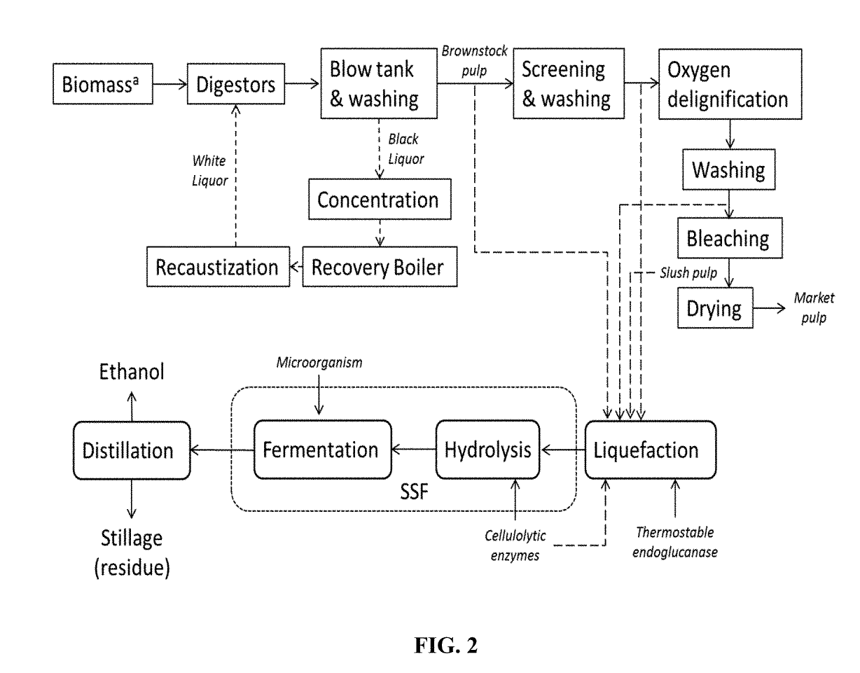

FIG. 2 illustrates another embodiment of a method of ethanol production from cellulosic pulp that is similar to the method illustrated in FIG. 1, but adds a liquefaction step before hydrolysis. Liquefaction can be carried out according to methods known in the art. For example, the cellulosic pulp may be treated with thermostable endoglucanase at 65 to 90° C. at a pH of 4.5 to 5.5. The cellulolytic enzymes can be added either during or after liquefaction, depending on whether the liquefaction conditions are conducive to the functioning of the cellulolytic enzymes.

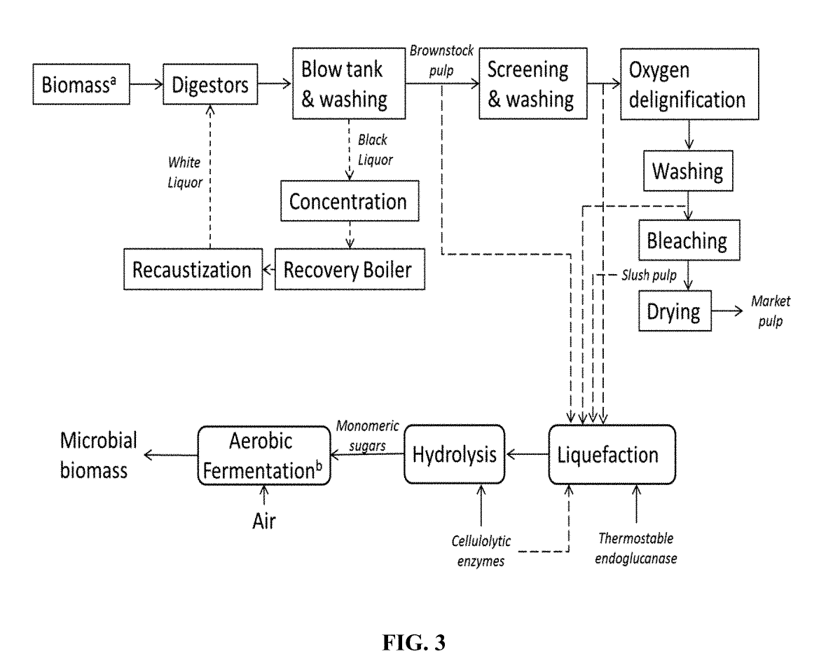

FIG. 3 illustrates an embodiment of a method of biomass production from cellulosic pulp. It is similar to the method illustrated in FIG. 2, but instead of performing fermentation simultaneously with hydrolysis, the monomeric sugars are collected and used to feed microbes in a process of aerobic fermentation, which can be a batch process or a fed-batch process, depending on the requirements of the microorganism being propagated. In both of the processes illustrated in FIG. 2 and FIG. 3, the liquefaction step is optional.

### E. Examples

### Example 1

### Production of Ethanol from Brownstock and Bleached Pulp with Sequential Hydrolysis and Fermentation

Cellulosic pulp, which is predominantly composed of glucan in the form of cellulose, can be effectively used in enzymatic hydrolysis followed by fermentation to produce biofuels (ethanol) or biochemicals. The sugar generated may also be subjected to a solid/liquid separation step and the sugar solution can be used for production of microbial biomass, e.g. yeast (that can go in food or feed applications) or heterotrophic algae (that can be used as fish feed in aquaculture).

Both brownstock and bleached pulp streams could be used for subsequent processing. However, using brownstock pulp can reduce the total amount of processing compared to bleached pulp, making it potentially more efficient for use in production of biofuels. However, bleached pulp may be better suited for production of relatively high-value biochemicals. Therefore, both brownstock pulp and bleached pulp were used in enzymatic hydrolysis and fermentation studies to see the level of ethanol that can be produced.

Initial work was done using brownstock pulp and bleached pulp obtained from a sulfite pulping mill. Upon receipt of the pulp samples, the percent total solids were measured by the oven drying method. The composition of the various pulp samples is set forth in Table 1. The percentages of the indicated components are given on a dry weight basis.

TABLE 1
Compositional analysis of various pulp samples
from a Kraft mill processing softwood

| | Arabinan | Galactan | Glucan | Xylan | Mannan | Lignin |
|---|---|---|---|---|---|---|
| | (% dry | (% dry | (% dry | (% dry | (% dry | (% dry |
| Sample | basis) | basis) | basis) | basis) | basis) | basis) |
| Brown- | 0.5 | 0.4 | 70.2 | 8.1 | 5.4 | 5.9 |
| stock | | | | | | |
| pulp | | | | | | |
| Washed | 0.5 | 0.4 | 74.5 | 8.5 | 5.7 | 4.3 |
| brown- | | | | | | |
| stock | | | | | | |
| pulp | | | | | | |
| Oxygen | 0.5 | 0.3 | 79.5 | 8.8 | 6.0 | 2.2 |
| de- | | | | | | |
| lignified | | | | | | |
| pulp | | | | | | |
| Bleached | 0.5 | 0.3 | 80.5 | 8.8 | 6.4 | 0.1 |
| pulp | | | | | | |

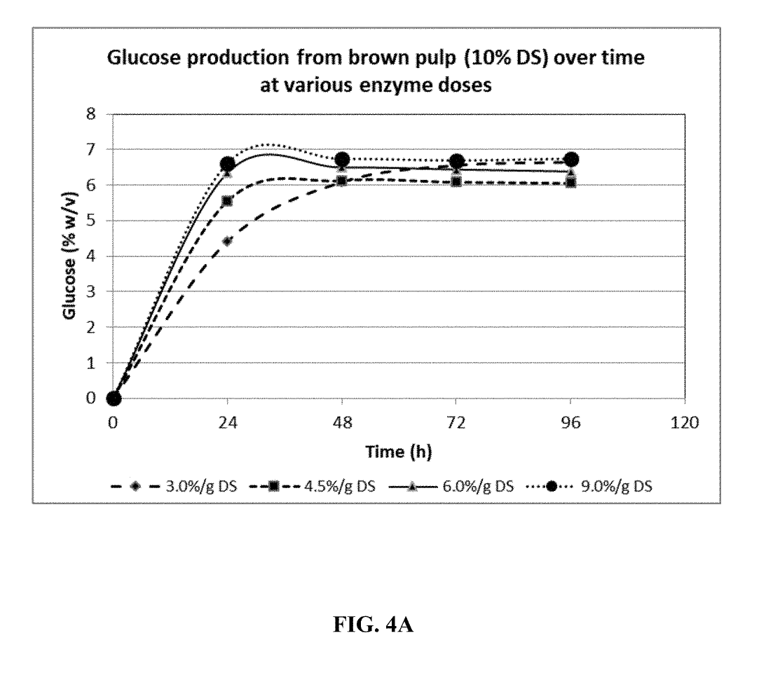

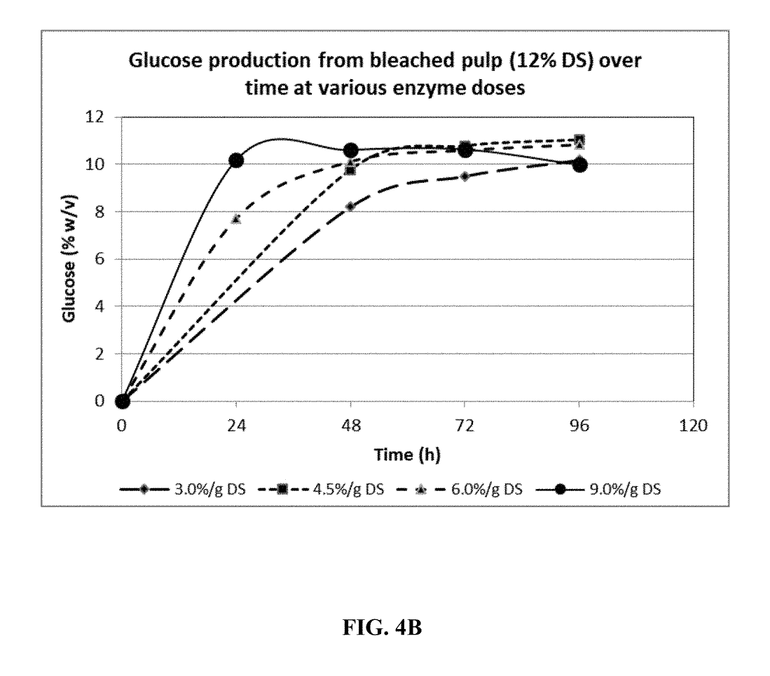

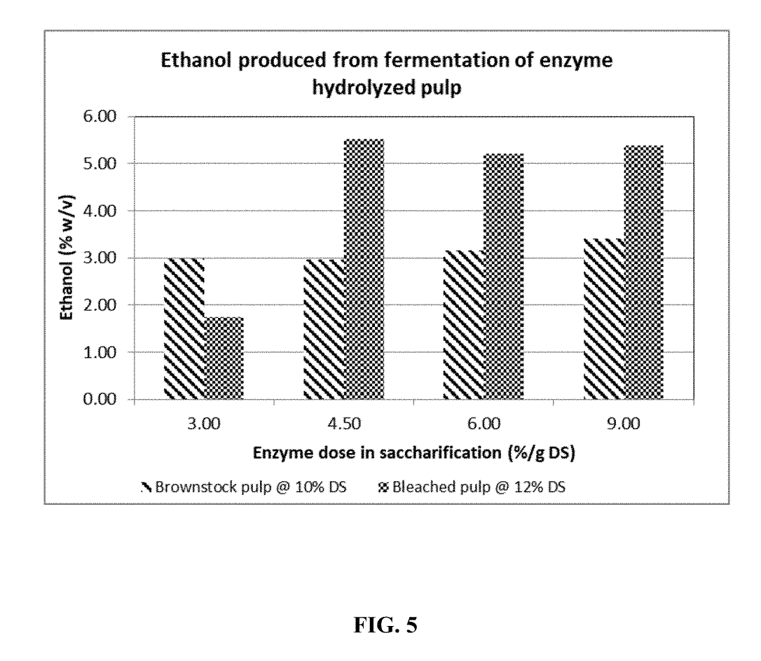

Based on the solids content of the pulp, the sequential hydrolysis/saccharification and fermentation (SHF) was set at 10% and 12% dry solids for the brownstock pulp and bleached pulp, respectively. The pulp sample was pH adjusted to 5.5 using 10% v/v sulfuric acid. The saccharifications were conducted in 100 mL media bottles with 50 g pulp slurry at the set solids levels. An enzyme dose response at different levels of cellulase cocktail, CTec3 HS from Novozymes was performed on each of the pulp samples. The enzyme doses chosen were 3%, 4.5%, 6% and 9% of enzyme product per gram dry solids. The saccharification was done at 50° C. in a water bath shaker (150 rpm) for 96 h. Samples were withdrawn every 24 h for 96 h and analyzed for sugars using a HPLC. After 96 h, the pulp samples were cooled to 32° C. and pH adjusted back to 5.5 for fermentation. Then, urea was added at 0.48 g/L as nitrogen source and non-genetically modified commercial Saccharomyces cerevisiae yeast strain was inoculated at 0.5 g/L. The fermentation was carried out at 32° C. in a water bath shaker (150 rpm) for 72 h. Samples were withdrawn at 72 h and analyzed for sugars, organic acids and alcohols using HPLC. The results from saccharification of brownstock pulp and bleached pulp are shown in FIGS. 4A and 4B, respectively. The ethanol production results are shown in FIG. 5.

The pulp samples hydrolyzed very well yielding good levels of glucose at all the enzyme dosages tested. Both the brownstock pulp and the bleached pulp samples showed good enzyme hydrolysis yields and ethanol yields.

Thusmilar work was carried out with pulp from 3 other mills, one using hardwood in a Kraft pulping process, one using southern softwood in a Kraft pulping process, and one using hardwood in a soda pulping process. Results are shown in Tables 2, 3, and 4, respectively.

TABLE 2
Glucose and ethanol after a sequential hydrolysis and fermentation
of pulp samples from hardwood in a Kraft pulping process.

| | Brownstock pulp | | Bleached pulp | |
|---|---|---|---|---|
| | @ 11% solids | | @ 11% solids | |
| Enzyme dose | Glucose^{a} | Ethanol^{b} | Glucose^{a} | Ethanol^{b} |
| (%/g DS) | (% w/v) | (% w/v) | (% w/v) | (% w/v) |
| 4.5 | 5.77 | 2.84 | 8.34 | 3.83 |
| 6.0 | 6.47 | 3.24 | 8.59 | 4.16 |

^{a}After 96 hours of saccharification at 50° C., initial pH adjusted to 5.5
^{b}After 48 hours of fermentation at 32° C., initial pH adjusted to 5.5

TABLE 3
Glucose and ethanol after a sequential hydrolysis and fermentation
of pulp samples from southern softwood in a Kraft pulping process.

| | Brownstock | | Bleached | |
|---|---|---|---|---|
| | pulp @ 12% solids | | pulp @ 9% solids | |
| Enzyme dose | Glucose^{a} | Ethanol^{b} | Glucose^{a} | Ethanol^{b} |
| (%/g DS) | (% w/v) | (% w/v) | (% w/v) | (% w/v) |
| 4.5 | 8.14 | 4.21 | 5.88 | 2.89 |
| 6.0 | 7.92 | 3.93 | 6.13 | 2.76 |

^{a}After 96 hours of saccharification at 50° C., initial pH adjusted to 5.5
^{b}After 48 hours of fermentation at 32° C., initial pH adjusted to 5.5

TABLE 4
Glucose and ethanol after a sequential hydrolysis and fermentation
of pulp samples from hardwood in a soda pulping process.

| | Brownstock | | Bleached | |
|---|---|---|---|---|
| | pulp @ 12% solids | | pulp @ 12% solids | |
| Enzyme dose | Glucose^{a} | Ethanol^{b} | Glucose^{a} | Ethanol^{b} |
| (%/g DS) | (% w/v) | (% w/v) | (% w/v) | (% w/v) |
| 4.5 | n/a | n/a | 6.62 | 3.00 |
| 6.0 | 7.64 | 3.62 | 6.75 | 3.06 |
| 9.0 | 7.77 | 3.60 | 7.09 | 3.36 |

^{a}After 96 hours of saccharification at 50° C., initial pH adjusted to 5.5
^{b}After 48 hours of fermentation at 32° C., initial pH adjusted to 5.5
n/a—not applicable

### EXAMPLE 2

### Production of Ethanol from Brownstock and Bleached Pulp with Simultaneous Hydrolysis and Fermentation

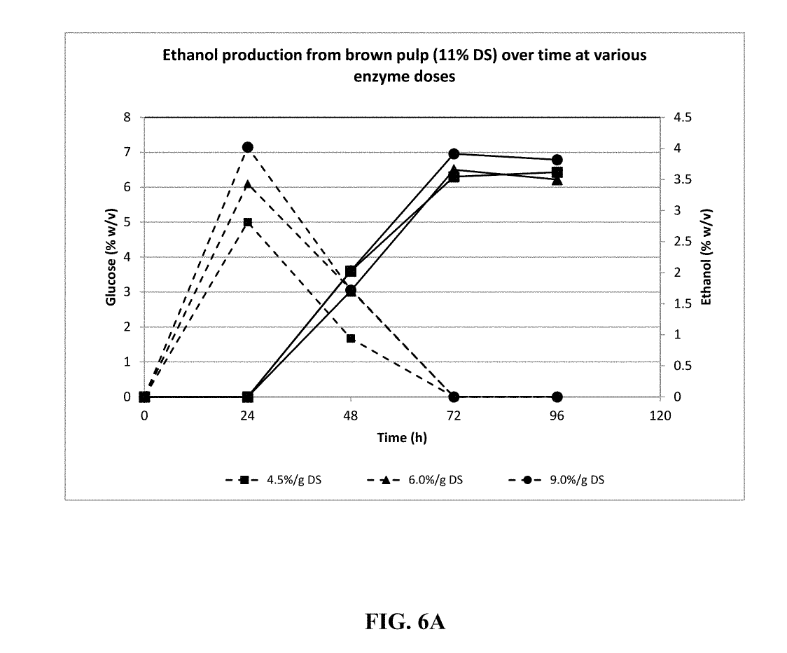

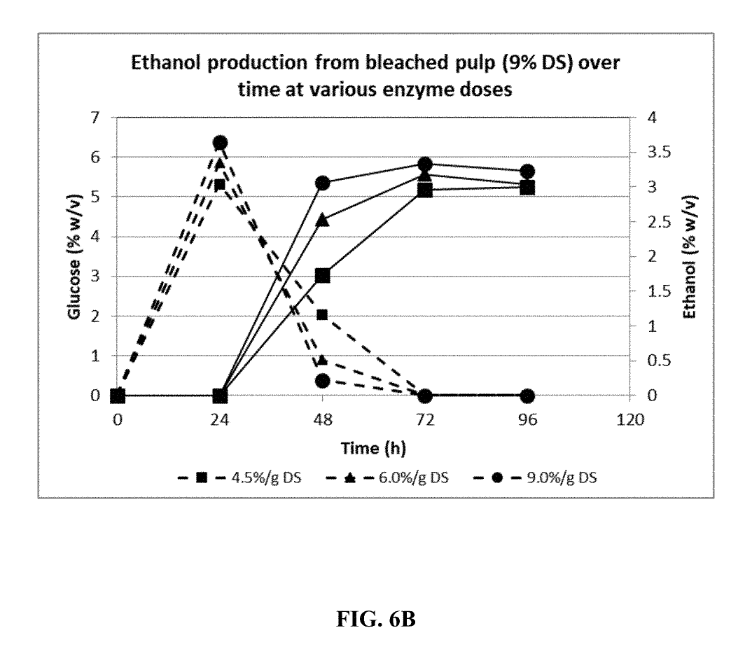

In order to reduce the overall time for saccharification and fermentation of pulp, a hybrid saccharification and fermentation study was performed. When scaled up, this would essentially be a short enzyme hydrolysis step (where the pulp is liquefied) followed by a simultaneous saccharification fermentation (SSF) step to produce ethanol. This work was done using brownstock pulp and bleached pulp from southern softwood in a Kraft pulping process. Based on the solids content of the pulp samples, the hybrid saccharification and fermentation was set at 11% and 9% dry solids for the brownstock pulp and bleached pulp, respectively. The pulp sample was pH adjusted to 5.5 using 10% v/v sulfuric acid. The study was conducted in 100 mL media bottles with 50 g pulp slurry at the set solids levels. Three enzyme doses—3%, 4.5%, 6% and 9% of CTec3 HS per g dry solids were chosen. The initial hydrolysis/saccharification was done at 50° C. in a water bath shaker (150 rpm) for 24 h. Samples were withdrawn at 24 h and analyzed for sugars using HPLC. Then, the partially hydrolyzed pulp samples were cooled to 37° C. and pH adjusted back to 5.5 for a simultaneous saccharification and fermentation. Urea was added at 0.48 g/L as nitrogen source and non-genetically modified commercial Saccharomyces cerevisiae yeast strain was inoculated at 0.5 g/L. The SSF was carried out at 37° C. in a water bath shaker (150 rpm) for an additional 72 h. Samples were withdrawn every 24 h during the SSF process and analyzed for sugars, organic acids and alcohols using a HPLC. The results are shown in FIGS. 6A and 6B.

The above specification and examples provide a complete description of the implementation and structure of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary step or structure, and/or may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A method of producing an end product from pulp, the method comprising:
(a) obtaining an aqueous slurry comprising 8 to 12% by weight of cellulosic pulp;
(b) contacting the cellulosic pulp with an enzyme composition to produce fermentable sugars, wherein the aqueous slurry is at a temperature between 45 and 60° C. and a pH between 4.5 and 6; and
(c) using the fermentable sugars as a carbon source for a microorganism to produce an end product.

2. The method of claim 1, wherein the cellulosic pulp is brownstock pulp or bleached pulp from a kraft pulping process or a sulfite pulping process.

3. The method of claim 1, further comprising adjusting the temperature of the aqueous slurry to between 25 and 38° C. before or during step (c).

4. The method of claim 1, wherein the end product is ethanol and wherein step (c) further comprises fermentation of the fermentable sugars by the microorganism and production of ethanol to a concentration of at least 3% w/v in the aqueous slurry.

5. The method of claim 1, wherein the end product comprises one or more of the following biochemicals produced by the microorganism: acetic acid, lactic acid, succinic acid, citric acid, gluconic acid, L-ascorbic acid, or itaconic acid.

6. The method of claim 1, wherein enzymatic hydrolysis of the cellulosic pulp continues during at least a portion of step (c).

7. The method of claim 1, further comprising separating the fermentable sugars from solids in the aqueous slurry before step (c), wherein the end product comprises biomass of the microorganism.

8. The method of claim 7, wherein step (c) comprises aerobic propagation of the microorganism and does not comprise anaerobic fermentation of the sugars.

9. The method of claim 7, wherein no more than 0.1 g/L of ethanol is produced by the by the microorganism during step (c), and wherein the microorganism is Saccharomyces cerevisiae.

10. The method of claim 7, wherein step (c) results in an increase of the biomass of the microorganism by at least 10 fold.

11. The method of claim 1, wherein step (c) further comprises providing a nitrogen source to the microorganism.

12. The method of claim 1, wherein contacting the cellulosic pulp with the enzyme composition results in the production of glucose to a concentration of at least 5% w/v in the aqueous slurry.

13. The method of claim 1, wherein the microorganism is capable of metabolizing five-carbon sugars.

14. The method of claim 1, wherein the total weight of the enzyme composition is between 3% and 9% of the dry weight of the cellulosic pulp in the aqueous slurry.

15. The method of claim 1, wherein the enzyme composition comprises one or more enzymes selected from the group of enzymes consisting of an endoglucanase, a cellobiohydrolase, a xylanase, and a beta-glucosidase.

16. The method of claim 7, wherein step (c) further comprises providing a nitrogen source to the microorganism.

17. The method of claim 7, wherein contacting the cellulosic pulp with the enzyme composition results in the production of glucose to a concentration of at least 5% w/v in the aqueous slurry.

18. The method of claim 7, wherein the microorganism is capable of metabolizing five-carbon sugars.

19. The method of claim 7, wherein the total weight of the enzyme composition is between 3% and 9% of the dry weight of the cellulosic pulp in the aqueous slurry.

20. The method of claim 7, wherein the enzyme composition comprises one or more enzymes selected from the group of enzymes consisting of an endoglucanase, a cellobiohydrolase, a xylanase, and a beta-glucosidase.
